(19) United States
(12) Patent Application Publication
(10) Pub. No.: US 2024/0353421 A1
(43) Pub. Date: Oct. 24, 2024

(54) IMMUNOHISTOCHEMISTRY (IHC) MAGE-A4 SCORING PROTOCOLS AND METHODS FOR AIDING CANCER TREATMENTS

(71) Applicant: Agilent Technologies, Inc., Santa Clara, CA (US)

(72) Inventors: Sneha Latha Koneru, Goleta, CA (US); Andrea Marquez, Santa Clara, CA (US); Maria Barbara Guillergan, Santa Clara, CA (US); Kristopher Kersch, Santa Clara, CA (US); Bethany Wynne, Carpinteria, CA (US); Pedro Corona, Santa Clara, CA (US); Alexander Prenta, Carpinteria, CA (US); Christopher Laplaca, Santa Clara, CA (US); Gregoriy Smiyun, Santa Clara, CA (US)

(21) Appl. No.: 18/426,938

(22) Filed: Jan. 30, 2024

Related U.S. Application Data
(60) Provisional application No. 63/442,381, filed on Jan. 31, 2023.

(51) Int. Cl. G01N 33/68; C07K 16/30; G01N 1/28; G01N 1/30; G01N 33/574

(52) U.S. Cl. CPC G01N 33/6854; C07K 16/30; G01N 1/2813; G01N 1/30; G01N 33/57492

(57) ABSTRACT

In alternative embodiments, provided are immunohistochemistry (IHC) methods for determining and scoring reproducibly the extent of expression of the protein Melanoma Associated Antigen Gene-A4 (MAGE-A4), in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring MAGE-A4 expression and utilizing the score as a companion or complementary diagnostic or to treat or ameliorate cancer or a tumor.

### RELATED APPLICATIONS

This U.S. utility patent application claims the benefit of priority under 35 U.S.C. § 119(e) to U.S. Provisional Patent Application Ser. No. (USSN) 63/442,381, Jan. 31, 2023. The aforementioned application is expressly incorporated herein by reference in their entirety and for all purposes.

### TECHNICAL FIELD

This invention generally relates to cancer treatments, companion or complementary diagnostics and immunohistochemical methods. In alternative embodiments, provided are immunohistochemistry (IHC) methods for determining and scoring reproducibly the extent of expression of the protein Melanoma Associated Antigen Gene-A4 (MAGE-A4) in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring MAGE-A4 expression and utilizing the score as a companion or complementary diagnostic or to treat or ameliorate cancer or a tumor.

### BACKGROUND

Melanoma Associated Antigen Gene-A4 (MAGE-A4) is located at chromosomal location Xq28, and has been implicated in some hereditary disorders, such as dyskeratosis congenita. At least four variants encoding the same protein have been found for this gene.

MAGE-A4 is expressed in many cancers and tumors; for example, in synovial sarcomas, myxoid/round cell liposarcomas, and lung cancers. A targeted treatment using genetically modified autologous T cells directed against MAGE-A4 is undergoing clinical trials.

It can be confusing as to how to apply the scoring guidelines, since tumor morphology in some cancers is heterogeneous. There remains a need in the art for simpler and more efficient, as well as robust, reproducible, and accurate scoring methods to assess MAGE-A4 expression in tissue samples.

### SUMMARY

In alternative embodiments, provided are immunohistochemistry (IHC) methods for determining and scoring the extent of cellular expression of Melanoma Associated Antigen Gene-A4 (MAGE-A4) in a tissue sample, comprising:
(a) staining a tissue sample with an antibody which specifically binds to MAGE-A4;
(b) determining a total number of viable tumor or cancer cells having MAGE-A4 staining, and determining a total number of staining and non-staining viable tumor or cancer cells in at least a portion of the tissue sample,
wherein a tumor or cancer cell is counted as positively stained with
anti-MAGE-A4 antibody if there is cytoplasmic and/or nuclear MAGE-A4 staining at any intensity above a defined threshold; and
(c) determining a MAGE-A4 tumor intensity proportion score (MAGE-A4 TIPS),
wherein the MAGE-A4 TIPS is the number of MAGE-A4 staining viable tumor or cancer cells found in the tissue sample divided by the total number of staining and non-staining viable tumor or cancer cells, multiplied by 100.

In alternative embodiments of IHC methods as provided herein:
the defined threshold comprises:
a 1+ positive staining intensity evaluated at a high magnification;
a 2+ positive staining intensity evaluated at a medium magnification; or
a 3+ positive staining intensity evaluated at a low magnification;
the low magnification is at least about 4× magnification; the medium magnification is at least about 10× magnification; and the high magnification is at least about 20× magnification or at least about 40× magnification;
the MAGE-A4 TIPS comprises the number of MAGE-A4 viable tumor or cancer cells staining at a 2+ or greater positive staining intensity divided by the total number of staining and non-staining viable tumor or cancer cells, multiplied by 100;
the MAGE-A4 TIPS comprises the number of MAGE-A4 viable tumor or cancer cells staining at a 1+ or greater positive staining intensity divided by the total number of staining and non-staining viable tumor or cancer cells, multiplied by 100;
a MAGE-A4 TIPS of about 70% or greater indicates a positive diagnostic status of the tissue sample; or, the MAGE-A4 TIPS is about 75% or greater, about 80% or greater, or about 90% or greater;
a MAGE-A4 TIPS of about 5% or greater indicates a positive diagnostic status of the tissue sample; or, a MAGE-A4 TIPS of about 5% or greater comprises the number of MAGE-A4 viable tumor or cancer cells staining at a 1+ or greater positive staining intensity; or a MAGE-A4 TIPS of about 10% or greater comprises the number of MAGE-A4 viable tumor or cancer cells staining at a 2+ or greater positive staining intensity;
a section or portion of the tissue sample is prepared on a slide, a microscope slide, or equivalent, and the section or portion of the tissue sample is stained on the slide;
the antibody comprises a monoclonal mouse anti-MAGE-A4 antibody, and optionally the monoclonal mouse anti-MAGE-A4 antibody comprises:
   monoclonal mouse anti-MAGE-A4 clone OTI1F9 antibody (generated using full length human recombinant protein of human MAGE-A4 (NP_001011550) produced in HEK293T cell as immunogen) (Agilent Technologies), or an antibody having a substantially similar affinity for MAGE-A4;
   as a clone OTI1F9 antibody (Origene, Abcam), or an antibody having a substantially similar affinity for MAGE-A4;
   a monoclonal mouse anti-MAGE-A4 clone 57B antibody, or an antibody having a substantially similar affinity for MAGE-A4;
   a monoclonal mouse anti-MAGE-A4 clone 6C1 antibody, or an antibody having a substantially similar affinity for MAGE-A4;
   a monoclonal mouse anti-MAGE-A4 clone CPTC-MAGEA4-1 antibody (Developmental Studies Hybridoma Bank), or an antibody having a substantially similar affinity for MAGE-A4;
   a monoclonal mouse anti-MAGE-A4 clone CPTC-MAGEA4-2 antibody, or an antibody having a substantially similar affinity for MAGE-A4;
   a monoclonal mouse anti-MAGE-A4 clone 8BA20 antibody (Creative Biolabs), or an antibody having a substantially similar affinity for MAGE-A4;
   a monoclonal mouse anti-MAGE-A4 clone 8BA21 antibody (Creative Biolabs), or an antibody having a substantially similar affinity for MAGE-A4;
   a monoclonal mouse anti-MAGE-A4 clone CBFYM-1312 antibody (Creative Biolabs), or an antibody having a substantially similar affinity for MAGE-A4; or
   a monoclonal mouse anti-MAGE-A4 clone 9A7 antibody (Creative Biolabs), or an antibody having a substantially similar affinity for MAGE-A4; or
   any combination thereof,
the tissue sample comprises a formalin-fixed, paraffin-embedded (FFPE) specimen, and optionally the FFPE specimen comprises a cancer specimen stained on an automated IHC platform;
the section of the tissue sample is prepared by a protocol comprising fixation in about 10% neutral buffered formalin for a time period of about 6 hours to about 72 hours;
the tumor or cancer is a synovial sarcoma, a myxoid/round cell liposarcoma, a high grade myxoid liposarcoma, a low grade myxoid liposarcoma, a head and neck cancer, a melanoma, an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer;
the tissue sample is derived from a needle biopsy sample, a fine-needle aspirate, a cytology specimen, or a bone decalcification;
positive staining is determined using a bright-field light microscope, a microscope objective, a computer monitor and imaging software, or a combination thereof; and/or
the imaging software comprises whole slide imaging software.

In alternative embodiments, provided are methods for diagnosing a tumor or a cancer by determining if a tissue sample is positive for expression of Melanoma Associated Antigen Gene-A4 (MAGE-A4), comprising:
determining a MAGE-A4 positive diagnostic status in a tissue sample by a method as provided herein, wherein a MAGE-A4 TIPS of about 70% or greater of tumor cancer cells having MAGE-A4 nuclear and/or cytoplasmic staining at an intensity of 2+ or greater is diagnostically positive.

In alternative embodiments of methods for diagnosing a tumor or a cancer as provided herein: the tumor or cancer is a synovial sarcoma, a myxoid/round cell liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

In alternative embodiments, provided are methods for treating or ameliorating a tumor or a cancer in a patient, comprising determining and scoring the amount of MAGE-A4 in a tissue sample from the patient using a method as provided herein, wherein if the tissue sample is determined or scored to have a high or a diagnostically positive MAGE-A4 TIPS, the patient is treated with a cancer therapeutic to which the patient is likely to respond favorably.

In alternative embodiments of methods for treating or ameliorating a tumor or a cancer in a patient:
the tumor or cancer is a synovial sarcoma, a myxoid/round cell liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer;
the cancer therapeutic comprises administration to the patient an anti-cancer drug or an anti-cancer therapy; and/or
the anti-cancer therapy comprises an immunotherapy, a monoclonal antibody therapy, an adoptive cell therapy (ACT), a T-cell receptor (TCR) therapy, or a chimeric antigen receptor (CAR) T-cell therapy.

In alternative embodiments, provided are kits comprising an antibody which specifically binds to MAGE-A4 and MAGE-A4 scoring guidelines as set forth in a method as provided herein, or as set forth in a method for diagnosing a tumor or a cancer as provided herein.

In alternative embodiments, provided are methods for assessing the extent of MAGE-A4 expression comprising:
contacting a sample or a portion thereof comprising cancer or tumor cells from an individual with an antibody or a portion thereof which specifically binds to MAGE-A4; and
determining a MAGE-A4 tumor intensity proportion score (MAGE-A4 TIPS) by dividing the number of MAGE-A4 staining viable tumor or cancer cells in the sample or portion thereof specifically bound by the antibody with the total number of staining and non-staining viable cancer or tumor cells and multiplying the result by 100, thereby obtaining the MAGE-A4 TIPS.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

Like reference symbols in the various drawings indicate like elements.

FIG. 1 shows a synovial sarcoma tissue sample (Block ID: SB00046226) stained with MAGE-A4 IHC (SK032).

FIG. 2 shows a squamous non-small cell lung cancer (Case ID: 1408330B) tissue sample stained with MAGE-A4 IHC (SK032).

FIG. 3 shows scoring of MAGE-A4 slides by three qualified observers. Scoring was performed manually using a light microscope and digitally by viewing whole slide images (WSIs) in the APERIO IMAGESCOPE™ viewing software.

### DETAILED DESCRIPTION

In alternative embodiments, provided are immunohistochemistry (IHC) methods for determining and scoring the extent of cellular expression of the protein Melanoma Associated Antigen Gene-A4 (MAGE-A4) in a tissue sample. In alternative embodiments, provided are scoring methods to assess MAGE-A4 expression in tumors or cancers. In alternative embodiments, provided are methods for diagnosing and/or treating a tumor or a cancer comprising use of scoring methods as provided herein to assess MAGE-A4 expression and determine if a high or a diagnostically positive MAGE-A4 Tumor Intensity Proportion Score (MAGE-A4 TIPS) is present. In certain embodiments, if a tissue sample is determined or scored to have a high or a diagnostically positive MAGE-A4 TIPS, the patient is treated with a cancer therapeutic to which the patient is likely to respond favorably.

Synovial sarcoma (SS) is a rare malignancy with a poor prognosis and low response rates to a variety of classes of therapeutic agents. This warrants development of targeted biomarkers for patient selection for novel adoptive T-cell therapies (ACT), for which there is evidence of clinical efficacy in SS. Melanoma Associated Antigen Gene-A4 (MAGE-A4), a cancer testis antigen, is highly expressed in SS and correlated with advanced clinical stage. The restricted expression pattern of MAGE-A4 in normal tissue makes it an attractive target for development of diagnostic assays and for ACT.

Embodiments herein are directed to immunohistochemistry methods for determining and scoring the extent of nuclear and/or cytoplasmic expression of MAGE-A4 in a tissue sample. In various embodiments, the method comprises: staining a tissue sample with an antibody which specifically binds to MAGE-A4; determining a total number of viable tumor or cancer cells having MAGE-A4 nuclear and/or cytoplasmic staining, and determining a total number of staining and non-staining viable tumor or cancer cells in at least a portion of a tissue sample, wherein a tumor or cancer cell is counted as positively stained with anti-MAGE-A4 antibody if there is MAGE-A4 cytoplasmic and/or nuclear staining at any intensity above a defined threshold; and determining a MAGE-A4 tumor intensity proportion score (MAGE-A4 TIPS), wherein the MAGE-A4 TIPS is the number of MAGE-A4 staining viable tumor or cancer cells found in the tissue sample divided by the total number of staining and non-staining viable tumor or cancer cells, multiplied by 100. Such embodiments can provide not only robust, reliable, reproducible and accurate MAGE-A4 scoring methods, but also scoring methods having greater simplicity and efficiency. Such embodiments can provide a considerable advantage for reliable and efficient diagnostic assessments.

In certain embodiments, the tissue sample comprises at least about 50 viable tumor or cancer cells. In certain embodiments, the tissue sample comprises at least about 100 viable tumor or cancer cells. In certain embodiments, the tissue sample comprises at least about 200 viable tumor or cancer cells. In certain embodiments, the tissue sample comprises at least about 500 viable tumor or cancer cells.

In some embodiments, a tumor or cancer cell counted as positively stained excludes staining of normal or non-neoplastic structures, staining nonviable tumor or cancer cells, necrotic cells, cellular debris, stromal staining, staining immune cells, staining benign cells, or edge artifact staining on a periphery of the tissue sample. In certain embodiments, a tumor or cancer cell counted as positively stained excludes tumor or cancer cells staining at less than 2+ positive staining intensity. Such embodiments can provide advantages of increasing the accuracy and reproducibility of MAGE-A4 scoring methods by excluding stained cells having certain structures or characteristics from the MAGE-A4 TIPS determination.

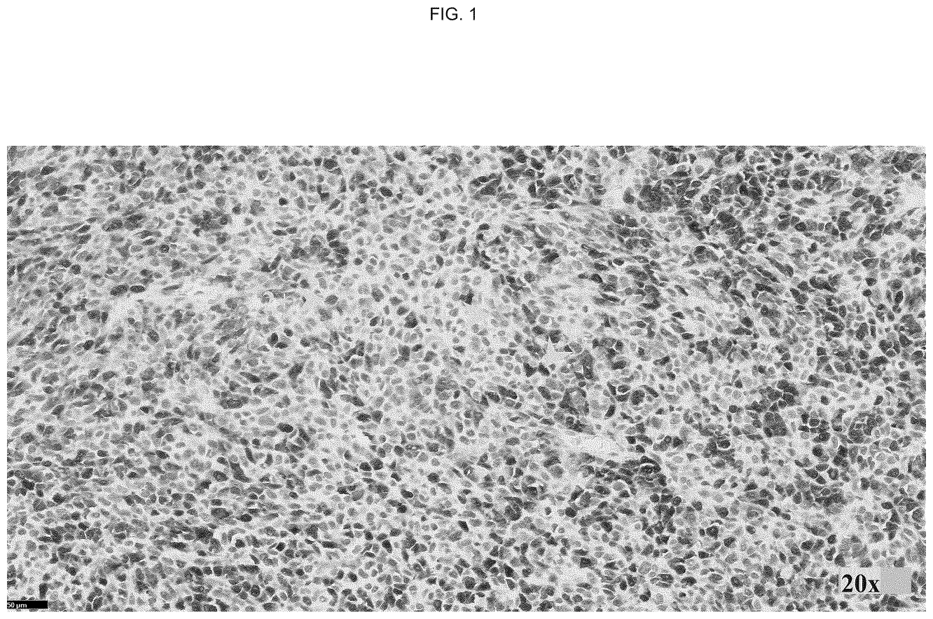

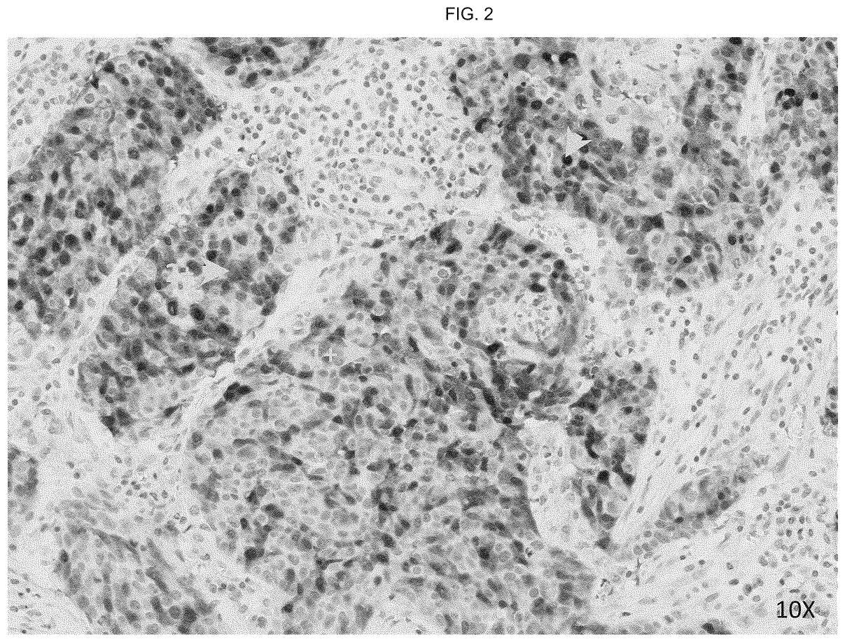

In certain embodiments, the defined threshold comprises a 1+ positive staining intensity evaluated at a high magnification. In certain embodiments, the defined threshold comprises a 2+ or greater positive staining intensity evaluated at a medium magnification. In certain embodiments, the defined threshold comprises a 3+ positive staining intensity evaluated at a low magnification (see FIG. 1 and FIG. 2). In some aspects, the low magnification is at least about 4× magnification; the medium magnification is at least about 10× magnification; or the high magnification is at least about 20× magnification or at least about 40× magnification.

In certain embodiments, the MAGE-A4 TIPS comprises the number of MAGE-A4 viable tumor or cancer cells staining at a 2+ or greater positive staining intensity divided by the total number of staining and non-staining viable tumor or cancer cells, multiplied by 100. In other embodiments, the MAGE-A4 TIPS comprises the number of MAGE-A4 viable tumor or cancer cells staining at a 1+ or greater positive staining intensity divided by the total number of staining and non-staining viable tumor or cancer cells, multiplied by 100. Such embodiments can provide a benefit of versatility in the staining intensity of the defined threshold.

In certain embodiments, a MAGE-A4 TIPS of about 70% or greater indicates a positive diagnostic status of the tissue sample. In certain embodiments, the MAGE-A4 TIPS is about 75% or greater, about 80% or greater, or about 90% or greater. In certain embodiments, a MAGE-A4 TIPS of about 5% or greater indicates a positive diagnostic status of the tissue sample. MAGE-A4 TIPS In certain embodiments, a MAGE-A4 TIPS of about 5% or greater comprises the number of MAGE-A4 viable tumor or cancer cells staining at a 1+ or greater positive staining intensity; or a MAGE-A4 TIPS of about 10% or greater comprises the number of MAGE-A4 viable tumor or cancer cells staining at a 2+ or greater positive staining intensity. Such embodiments can provide a benefit of accuracy in a diagnostic status determination related to MAGE-A4 expression.

In alternative embodiments, methods and kits as provided herein are used for in vitro diagnostic uses. In alternative embodiments, the MAGE-A4 IHC as provided herein is an immunohistochemical (IHC) assay using an anti-MAGE-A4 antibody such as the monoclonal mouse anti-MAGE-A4 clone OTI1F9 antibody (generated using full length human recombinant protein of human MAGE-A4 (NP_001011550) produced in HEK293T cell as immunogen) (Agilent Technologies), or an antibody having a substantially similar affinity for MAGE-A4, in the detection of a MAGE-A4 protein in FFPE tissue samples. In alternative embodiments, the AUTOSTAINER LINK 48™ automated staining system utilizing FFPE tissue sections is used.

In alternative embodiments, the MAGE-A4 IHC methods as provided herein are used as an aid in identifying patients with a tumor or a cancer, for example, a synovial sarcoma, a myxoid/round cell liposarcoma, a high grade myxoid liposarcoma, a low grade myxoid liposarcoma, a head and neck cancer, a melanoma, an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

In alternative embodiments, methods for assessing the extent of MAGE-A4 expression comprise: contacting a sample or a portion thereof comprising cancer or tumor cells from an individual with an antibody or a portion thereof which specifically binds to MAGE-A4; and determining a MAGE-A4 Tumor Intensity Proportion Score (MAGE-A4 TIPS) by dividing the number of MAGE-A4 staining viable tumor or cancer cells in the sample or portion thereof specifically bound by the antibody with the total number of staining and non-staining viable cancer or tumor cells and multiplying the result by 100, thereby obtaining the MAGE-A4 TIPS.

In certain embodiments, a section or portion of the tissue sample is prepared on a slide, a microscope slide, or equivalent, and the section or portion of the tissue sample is stained on the slide. In certain embodiments, the tissue sample comprises a an FFPE specimen. In certain embodiments, the FFPE specimen comprises a cancer specimen stained on an automated IHC platform. In certain embodiments, the section of the tissue sample is prepared by a protocol comprising fixation in about 10% neutral buffered formalin for a time period of about 6 hours to about 72 hours. In certain embodiments, the tissue sample is derived from a needle biopsy sample, a fine-needle aspirate, a cytology specimen, or a bone decalcification.

In certain embodiments, the antibody comprises a monoclonal mouse anti-MAGE-A4 antibody or a monoclonal rabbit anti-MAGE-A4 antibody. In some embodiments, the monoclonal mouse anti-MAGE-A4 antibody comprises: a monoclonal mouse anti-MAGE-A4 clone OTI1F9 antibody or an antibody having a substantially similar affinity for MAGE-A4 as the clone OTI1F9 antibody; a monoclonal mouse anti-MAGE-A4 clone 57B antibody or an antibody having a substantially similar affinity for MAGE-A4; a monoclonal mouse anti-MAGE-A4 clone 6C1 antibody or an antibody having a substantially similar affinity for MAGE-A4; a monoclonal mouse anti-MAGE-A4 clone CPTC-MAGEA4-1 antibody or an antibody having a substantially similar affinity for MAGE-A4; a monoclonal mouse anti-MAGE-A4 clone CPTC-MAGEA4-2 antibody or an antibody having a substantially similar affinity for MAGE-A4; a monoclonal mouse anti-MAGE-A4 clone 8BA20 antibody or an antibody having a substantially similar affinity for MAGE-A4; a monoclonal mouse anti-MAGE-A4 clone 8BA21 antibody or an antibody having a substantially similar affinity for MAGE-A4; a monoclonal mouse anti-MAGE-A4 clone CBFYM-1312 antibody or an antibody having a substantially similar affinity for MAGE-A4; or, monoclonal mouse anti-MAGE-A4 clone 9A7 antibody or an antibody having a substantially similar affinity for MAGE-A4; or, any combination thereof.

In certain embodiments, the tumor or cancer is a synovial sarcoma, a myxoid/round cell liposarcoma, a high grade myxoid liposarcoma, a low grade myxoid liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

In certain embodiments, the positive staining is determined using a bright-field light microscope, a microscope objective, a computer monitor and imaging software, or a combination thereof. In some aspects, the imaging software comprises whole slide imaging software.

### Embodiments of Methods for Diagnosing a Tumor or a Cancer

Embodiments herein provide methods for diagnosing a tumor or a cancer by determining if a tissue sample is positive for cellular expression of MAGE-A4. In various aspects, the method comprises: determining a MAGE-A4 diagnostic status in a tissue sample by an IHC method for determining the extent of cytoplasmic and/or nuclear expression of MAGE-A4 as provided herein, wherein a MAGE-A4 TIPS of about 70% or greater of tumor cancer cells having MAGE-A4 nuclear and/or cytoplasmic staining at an intensity of 2+ or greater is diagnostically positive. In certain embodiments, the tumor or cancer is a synovial sarcoma, a myxoid/round cell liposarcoma, a high grade myxoid liposarcoma, a low grade myxoid liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

### Embodiments of Methods for Treating Cancers and Tumors

Provided are methods for treating or ameliorating a tumor or a cancer in a patient, comprising determining and scoring the amount of MAGE-A4 in a tissue sample from the patient using a method as provided herein, wherein if the tissue sample is determined or scored to have a high or a diagnostically positive MAGE-A4 score, the patient is treated with a cancer therapeutic or an anti-cancer therapy to which the patient is likely to respond favorably. The tumor or cancer can be a synovial sarcoma, a myxoid/round cell liposarcoma, a high grade myxoid liposarcoma, a low grade myxoid liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

In certain embodiments, the cancer therapeutic comprises administration to the patient of an anti-cancer drug or an anti-cancer therapy. In alternative embodiments, the anti-cancer treatment or therapy comprises: surgery such as cyberknife therapy; chemo-embolization; an ablation technique such as radiofrequency ablation (RFA), cryoablation and/or microwave ablation; and/or, radiation therapy such as stereotactic body radiation therapy.

In certain embodiments, the anti-cancer therapy comprises an antibody drug conjugate, a small molecule therapy, an immunotherapy, a monoclonal antibody therapy, an adoptive cell therapy (ACT), a T-cell receptor (TCR) therapy, or a chimeric antigen receptor (CAR) T-cell therapy. In alternative embodiments, the anti-cancer treatment or therapy comprises: a tyrosine kinase inhibitor (optionally erlotinib (or TARCEVA™), gefitinib (or IRESSA™), afatinib (or GILOTRIF™), or osimertinib (TAGRISSO™)); necitumumab (or PORTRAZZA™), pembrolizumab (or KEYTRUDA™), nivolumab (or OPDIVO™), ipilimumab (YERVOY™) cetuximab (or ERBITUX™), cisplatin (or PLATINOL™) or carboplatin (or PARAPLATIN™); gemcitabine (or GEMZAR™), docetaxel (or TAXOTERE™) olaratumab (or LARTRUVO™), Doxorubicin (or ADRIAMYCIN™ or RUBEX™) pazopanib (or VOTRIENT™), ifosfamide (or IFEX™), trabectedin (or YONDELIS™).

In alternative embodiments, the anti-cancer treatment or therapy comprises use of an anti-cancer drug that can comprise an antibody that specifically or substantially binds to the cancer or tumor, wherein the antibody is conjugated to a cytotoxic agent, and optionally the cytotoxic agent comprises a radionuclide (optionally Yttrium-90, Iodine-131, Lutetium-177, Radium-223 chloride, strontium-89 chloride or samarium-153 EDTMP), diphtheria toxin, pseudomonas exotoxin A, denileukin diftitox, moxetumomab pasudotox, calicheamicin or N-acetyl-γ-calicheamicin, emtansine or DM1, maytansine or derivatives thereof, SN-38 (or 7-Ethyl-10-hydroxycamptothecin), or auristatin or monomethyl auristatin E (MMAE).

### Immunohistochemistry

In alternative embodiments, immunohistochemistry methodologies and/or reagents used with methods and products of manufacture or kits as provided herein can include or comprise or comprise use of any IHC protocol, IHC armamentarium, device and/or image or data analysis system, for practicing IHC or IHC reagents known in the art.

In alternative embodiments, the antibodies, antigen binding fragments thereof, or monomeric or dimeric antigen binding proteins as provided herein (including for example synthetic or recombinant forms) used in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, an IHC protocol or reagents.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase, for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-1-0-carboxylic acid; 7-(diethylamino)coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester; DY-405; Alexa Fluor 405 succinimidyl ester; Cascade Yellow succinimidyl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide; Alexa Fluor 430 succinimidyl ester; Dabcyl succinimidyl ester; NBD chloride/fluoride; QSY 35 succinimidyl ester; DY-485XL; Cy2 succinimidyl ester; DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505; Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL; DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester; DY-555; DYQ-1; DY-556; Cy3 succinimidyl ester; DY-547; DY-549; DY-550; Alexa Fluor 555 succinimidyl ester; Alexa Fluor 546 succinimidyl ester; DY-548; BODIPY 558/568 C3 succinimidyl ester; Rhodamine red-X succinimidyl ester; QSY 7 succinimidyl ester; BODIPY 564/570 C3 succinimidyl ester; BODIPY 576/589 C3 succinimidyl ester; carboxy-X-rhodamine (ROX); succinimidyl ester; Alexa Fluor 568 succinimidyl ester; DY-590; BODIPY 581/591 C3 succinimidyl ester; DY-591; BODIPY TR-X succinimidyl ester; Alexa Fluor 594 succinimidyl ester; DY-594; carboxynaphthofluorescein succinimidyl ester; DY-605; DY-610; Alexa Fluor 610 succinimidyl ester; DY-615; BODIPY 630/650-X succinimidyl ester; erioglaucine; Alexa Fluor 633 succinimidyl ester; Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701; DY-704; DY-700; DY-730; DY-731; DY-732; DY-734; DY-750; Cy7 succinimidyl ester; DY-749; DYQ-4; Cy7.5 succinimidyl ester; 7-diethylaminocoumarin-3-carboxylic acid; succinimidyl ester; Dabsyl sulfonyl chloride; fluorescein isothiocyanate (FITC) carboxy succinimidyl ester (DY-495); Rhodamine Green carboxylic acid succinimidyl ester (DY-505); eosin isothiocyanate (EITC); 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye. 3,3′-Diaminobenzidine (DAB) also can be used.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, MAGE-A4 protein), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or FFPE tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 μm to 5 μm. The sections or slices can be mounted on slides, dehydrated using alcohol washes of increasing concentrations (for example, 50%, 75%, 90%, 95%, 100%), and cleared using a detergent like xylene before being imaged or evaluated under a microscope.

Depending on the method of fixation and tissue preservation, the sample may require additional steps to make a MAGE-A4 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using EnVision FLEX Mini Kit, High pH, which is a high-sensitivity visualization system intended for use in IHC together with Dako AUTOSTAINER™ instruments; this dual link system detects primary mouse and rabbit antibodies and the reaction is visualized by 3,3-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein, including for example, at least one anti-MAGE-A4 antibody, for example, a monoclonal antibody, for example, a monoclonal mouse anti-MAGE-A4 clone OTI1F9 antibody (generated using full length human recombinant protein of human MAGE-A4 produced in HEK293T cell as immunogen) (Agilent Technologies), or an antibody having a substantially similar affinity for MAGE-A4, and/or reagents for practicing IHC, including for example, reagents as described herein, see Example 1, below; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of any patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK).

### Example 1: Exemplary IHC Protocols for Detecting MAGE-A4 Protein

This example describes exemplary IHC methods as provided herein, and demonstrates the efficacy of MAGE-A4-detecting IHC protocols as provided herein.

The results of the activities performed are presented as required to establish inter- and intra-observer scoring precision of FFPE synovial sarcoma (SS), myxoid round cell liposarcoma (MRCLS), and squamous cell carcinoma non-small cell lung cancer (sqNSCLC) specimens stained with MAGE-A4 IHC (SK032) assay on AUTOSTAINER LINK 48™. Specimens were evaluated using MAGE-A4 IHC Scoring Guidelines.

Verification of inter- and intra-observer precision was tested for SS, MRCLS, and sqNSCLC specimens using the MAGE-A4 IHC scoring guidelines. Specimens were stained using a MAGE-A4 IHC assay. The results of this report inform on the reproducibility in scoring of the assay on stained SS, MRCLS, and sqNSCLC specimens when scored multiple times by each of multiple observers.

### Reagents

MAGE-A4 IHC (SK032) contains optimized reagents with the protocol required to complete an IHC staining procedure on FFPE specimens using the PT Link Pre-Treatment Module™ (https://www.agilent.com/en/product/pt-link-for-pre-treatment/pt-link-accessories/pt-link-pre-treatment-module-for-tissue-specimens-76929) and AUTOSTAINER LINK 48™. Specimens are first incubated with Peroxidase-Blocking Reagent. Following incubation with the primary monoclonal antibody to MAGE-A4 or the Negative Control Reagent (NCR), specimens are incubated with the primary antibody, and then are incubated with a ready-to-use (RTU) visualization reagent consisting of secondary antibody molecules and horseradish peroxidase molecules coupled to a dextran polymer backbone. The enzymatic conversion of the subsequently added chromogen results in precipitation of a visible reaction product at the antigen site. The specimen may then be counterstained and coverslipped. Results are interpreted using a bright-field microscope.

TABLE 1
Assay components for exemplary
MAGE-A4-detecting IHC protocol

| Component | Component Description |
|---|---|
| Peroxidase-Blocking Reagent | Buffered solution containing |
| | hydrogen peroxide, detergent, |
| | and 0.015 mol/L sodium azide. |
| Mouse Monoclonal anti-MAGE- | Monoclonal Mouse anti-MAGE-A4 |
| A4 antibody, Clone OTI1F9 | in a buffered solution, |
| | containing stabilizing protein, |
| | and 0.015 mol/L sodium azide. |
| Negative Control Reagent (NCR) | Monoclonal Mouse control IgG2a |
| | antibody in a buffered solution, |
| | containing stabilizing protein, |
| | and 0.015 mol/L sodium azide. |
| Visualization Reagent-HRP | Dextran coupled with peroxidase |
| | molecules and goat secondary |
| | antibody molecules against |
| | rabbit and mouse immunoglobulins |
| | in a buffered solution containing |
| | stabilizing protein and an |
| | antimicrobial agent. |
| DAB + (3,3′-Diaminobenzidine) | Buffered solution, containing |
| Substrate Buffer | hydrogen peroxide and an |
| | antimicrobial agent. |
| DAB + Chromogen | 3,3′-diaminobenzidine |
| | tetrahydrochloride in organic solvent. |
| Target Retrieval Solution, High | Buffered solution, containing |
| pH, 50X | detergent and an antimicrobial agent. |

EnVision FLEX Hematoxylin (Dako K8008)
Automated coverslipping instrument or ClearMount (American MasterTech Scientific, MMCLE126), or equivalent mounting medium
Alcohol Reagent, Ethanol: absolute (Dako 100188) and 95%
Xylene (Dako 100941) or xylene substitute, i.e. Histoclear (Dako 100791)
EnVision FLEX Wash Buffer 20× (Dako K8007)
Distilled or deionized (reagent-grade water), or Dako reagent water (Dako PD00004)

### Reagent Preparation

Equilibrated kit reagents, buffers, and unstained slides to room temperature, 20-25° C.
Prepared 1× Wash Buffer by diluting 20× Wash Buffer in distilled or deionized (reagent-grade) water and mixing.
Discard Wash Buffer if it is cloudy in appearance.
Prepared 1× High pH Target Retrieval Solution (TRS) by diluting 50× High pH Target Retrieval Solution in reagent-grade water and mixing.
   Prepared a sufficient quantity of High pH 1× Target Retrieval Solution by diluting Target Retrieval Solution, High pH (50×) 1:50 using distilled or deionized (reagent-grade) water. One 30 mL bottle of Target Retrieval Solution, High pH (50×), diluted 1:50 provided 1.5 L of 1× reagent, sufficient to fill one PT Link tank which can treat up to 24 slides per use. TRS pH test should be performed to ensure 1×TRS is within reagent specifications (pH 9.0±0.2) prior to use.
   Prepared DAB+ chromogen shortly before initiating AUTOSTAINER LINK 48™ run. To each 1 mL of Substrate Buffer add 1 drop of Liquid DAB+ chromogen.
   If using a bottle of 7.2 mL DAB+ Substrate Buffer, add 9 drops of DAB+ chromogen. Although the label states 7.2 mL, this is the useable volume and does not account for the “dead volume” (1.8 mL) in the bottle.
   The color of the Liquid DAB+ Chromogen in the bottle may vary from clear to lavender-brown. This will not affect the performance of this product. Dilute per the guidelines above. Addition of excess Liquid DAB+ Chromogen to the DAB+ Substrate Buffer will result in deterioration of the positive signal.

### Specimen Preparation

All specimens were fixed in neutral buffered formalin and embedded in paraffin blocks. FFPE blocks were cut at 4 μm, mounted onto Dako FLEX™ IHC Microscope Slides and placed at 58 (±2) ° C. for approximately 1 hour within 18 hours of sectioning, then cooled to room temperature (RT). Cut sections were kept at 2-8° C. until use.

### Staining Procedure

### Deparaffinization/Rehydration/Target Retrieval Procedure: PT Link

All specimens were processed using the PT Link with the Preheat and Cool temperature set to 65° C. All specimens were then processed using a PT Link set Heat temperature to 97° C. for 20 minutes.
Filled PT Link tanks with 1.5 L per tank of Target Retrieval Solution, High pH, 1× working solution to cover the tissue sections.
Preheated the 1× Target Retrieval Solution to 65° C.
Immersed AUTOSTAINER LINK 48™ racks containing mounted, FFPE tissue sections into the pre-heated Target Retrieval Solution, High pH, (1× working solution) in PT Link tank. Incubate for 20 minutes at 97° C.
As soon as target retrieval incubation time has been completed and the temperature had cooled to 65° C., removed each AUTOSTAINER LINK 48™ slide rack with the slides from the PT Link tank and immediately placed the AUTOSTAINER LINK 48™ rack with slides into a tank (e.g., PT Link Rinse Station, Code PT109) containing 1×, room temperature EnVision FLEX™ Wash Buffer (Code K8007).
Immersed slides in diluted, room temperature 1× Wash Buffer for 5 minutes
   After deparaffinization, rehydration and target retrieval (3-in-1) procedure (specimen pre-treatment), the Autostainer racks with slides were placed on AUTOSTAINER LINK 48™. The AUTOSTAINER LINK 48™ instrument performed the staining process automatically using pre-programmed protocol by applying the appropriate reagent, monitoring the incubation time and rinsing slides between reagents.

### Data Interpretation

The following information was captured during observer reads:
MAGE-A4 slide: Percent positive MAGE-A4 score at each intensity bin (0 to 3+)
Diagnostic outcome
   Positive: MAGE-A4 TIPS≥2+ staining intensity at ≥75% cutoff
   Negative: MAGE-A4 TIPS≥2+ staining intensity at <75% cutoff
Average IHC staining intesity
NCR slide: Pass/Fail
   The Negative Control Reagent (NCR) stained slide must not display specific staining. Non-specific staining at ≤1+ staining intensity is acceptable.
   Specific staining is defined as nuclear and/or cytoplasmic staining in tumor cells and non-specific staining is defined as staining of non-tumor tissue elements. If the NCR specimen displays specific staining at any intensity, or non-specific staining at >1+ intensity, DO NOT proceed with evaluation of the MAGE-A4 stained slide
Comments, if applicable

### Scoring Guidelines

Stained slides for all indications were scored by qualified observers

### Specimen

Eighty specimens (sqNSCLC, MRCLS and/or SS) demonstrating a range of MAGE-A4 expression were selected in the study. Efforts were made to ensure a balanced positive/negative specimen set around the MAGE-A4 TIPS≥2+ staining intensity at ≥75% cut-off with approximately 20-25% of samples falling within the near cut-off range (65-85%). Stained slides were selected from previously performed screening runs and the methods as described herein were followed. FIG. 1 shows a Synovial sarcoma tissue sample (Block ID: SB00046226) stained with MAGE-A4 IHC (SK032). Arrows designate 1+, 2+, or 3+ staining intensities observed at 20× magnification.

FIG. 2 shows a squamous non-small cell lung cancer (Case ID: 1408330B) tissue sample stained with MAGE-A4 IHC (SK032). Arrows designate 1+, 2+, or 3+ staining intensities observed at 10× magnification. The data and biospecimens used in this project was provided by Contract Research Ltd (Charlestown, Nevis) with appropriate ethics approval and through Azenta Life Sciences.

### Procedure

Eighty stained specimens were evaluated by three observers (inter-observer) and were scored three times by each observer (intra-observer). A washout period of at least 14 days occurred between each observer reading session to minimize scoring recall bias.

All slides for all reads were blinded and randomized. Round labels were applied to each slide in accordance with the randomization scheme generated in Excel using the RAND function. Each intra-observer read had a different randomization scheme. The observers scoring for this study did not participate in the blinding and randomization of slides.

### Test Objections and Procedures

The objective of this test is to determine the scoring precision among multiple observers.

A blinded and randomized set of stained slides were provided in turn to each of three observers, with three reads by each observer. This resulted in nine scores for each case. The scores for each specimen were analyzed to determine the level of diagnostic agreement between multiple observers across multiple reads. The same set of data were used for both inter-observer and intra-observer analysis.

### Statistical Analysis Plan

### Calculation of Percent Agreements (NPA, PPA, and OA)

Percent agreement calculations were devised to compare each read from each observer against consensus. Consensus was defined as the most frequent diagnostic outcome observed.

### Confidence Interval on Percent Agreements

Two-sided 95% Confidence intervals for NPA, PPA, and OA were computed using bootstrap, which is a well-known computational technique that creates new datasets from the entire original dataset using random selection so that statistical uncertainty can be computed for creating statistical confidence intervals. In the event that NPA, PPA, or OA exactly equal 100%, bootstrap is known to fail, so Wilson score confidence intervals were computed.

### Acceptance Criteria

The lower bound of the two-sided 95% confidence interval calculated on each agreement parameter (NPA, PPA, and OA) must be ≥85%. Any additional analysis that is performed is not subject to acceptance criteria.

### Intra-Observer Precision

### Test Objective

The objective of this test was to determine the scoring precision within individual observers across multiple reads of the same sample.

### Test Procedure

A blinded and randomized set of stained slides were provided in turns to each of three observers, with three reads taken by each observer. This resulted in nine scores for each case. A washout period of at least 14 days occurred between each intra-observer read. The scores for each specimen were analyzed to determine the level of diagnostic agreement between multiple reads for individual observers. Note that the same set of data were used for both inter-observer and intra-observer analysis.

### Statistical Analysis Plan

Percent agreement calculations were devised to compare each read from each observer against consensus. Consensus was defined as the most frequent diagnostic outcome observed. Two-sided 95% Confidence intervals for NPA, PPA, and OA were computed using bootstrap, which is a well-known computational technique that creates new datasets from the entire original dataset using random selection so that statistical uncertainty can be computed for creating statistical confidence intervals for PPA, NPA, and OA. In the event that NPA, PPA, or OA exactly equal 100%, bootstrap is known to fail, so Wilson score confidence intervals were computed.

### Acceptance Criteria

The study will be accepted once all sub-studies have been completed, discrepancies (if present) addressed and resolved, and the final report is approved.

### Results

Data from all indications were combined for analysis and is presented in Table 2 and 3. Additional analysis per indication was performed for informational purposes

TABLE 2
Inter-observer results summary
MAGE-A4 IHC SS, MRCLS, NSCLC, Inter-Observer, MAGE-A4 TIPS
at ≥2 + staining intensity ≥ 75% Cut-off
Agreement Summary

| | | | 95% Confidence Interval | |
|---|---|---|---|---|
| | Performance | Point | Lower-bound: | Upper-bound: |
| Indication | Criteria | Estimate | 2.5% | 97.5% |
| MRCLS, | NPA | 94.3% | 89.6% | 98.0% |
| sqNSCLC, | PPA | 95.7% | 91.7% | 99.1% |
| SS combined | OA | 95.1% | 92.4% | 97.6% |
| MRCLS | NPA | 100.0%* | 90.4% | 100.0% |
| | PPA | N/A | N/A | N/A |
| | OA | 100.0%* | 90.4% | 100.0% |
| NSCLC | NPA | 93.1% | 86.8% | 97.9% |
| | PPA | 95.4% | 89.8% | 100.0% |
| | OA | 94.4% | 90.3% | 97.8% |
| SS | NPA | 94.0% | 84.6% | 100.0% |
| | PPA | 96.1% | 90.3% | 100.0% |
| | OA | 95.4% | 90.7% | 99.1% |

*The bootstrap method cannot compute confidence bounds if 100% agreement is observed. The Wilson Score limits are used to calculate confidence intervals for agreement parameters with point estimates equal to 100%.

TABLE 3
Intra-observer results summary
MAGE-A4 IHC SS, MRCLS, sqNSCLC, Intra-Observer, MAGE-A4 TIPS
at ≥2 + staining intensity ≥ 75% Cut-off
Agreement Summary

| | | | 95% Confidence Interval | |
|---|---|---|---|---|
| | Performance | Point | Lower-bound: | Upper-bound: |
| Indication | Criteria | Estimate | 2.5% | 97.5% |
| MRCLS, | NPA | 99.0% | 97.7% | 100.0% |
| sqNSCLC, | PPA | 99.1% | 98.0% | 99.8% |
| SS combined | OA | 99.0% | 98.3% | 99.6% |
| MRCLS | NPA | 100.0%* | 90.4% | 100.0% |
| | PPA | N/A | N/A | N/A |
| | OA | 100.0%* | 90.4% | 100.0% |
| NSCLC | NPA | 98.6% | 96.6% | 100.0% |
| | PPA | 99.1% | 97.6% | 100.0% |
| | OA | 98.9% | 97.8% | 99.7% |
| SS | NPA | 99.1% | 97.2% | 100.0% |
| | PPA | 99.0% | 97.5% | 100.0% |
| | OA | 99.1% | 97.8% | 100.0% |

*The bootstrap method cannot compute confidence bounds if 100% agreement is observed. The Wilson Score limits are used to calculate confidence intervals for agreement parameters with point estimates equal to 100%.

### Conclusions

The analyses for Intra-Observer and Inter-Observer precision showed that the lower bound of the two-sided 95% confidence intervals for NPA, PPA and OA for both Inter-Observer and Intra-Observer were at least 85%. Thus, statistically both Intra-Observer Precision and Inter-Observer Precision met the acceptance criteria. NPA, PPA, and OA were computed using a cutoff of 75% at ≥2+ staining intensity for MAGE-A4 TIPS. Inter/intra-observer precision has been established between/within individual observers across multiple reads of the same sample for SS, MRCLS, and sqNSCLC slides stained with the SK032 MAGE-A4 IHC assay when using the scoring system specified in this report. Additional analyses performed on individual indication data, were not subject to acceptance criteria.

Each of the three individual indications showed high agreement (point estimates >93%), suggesting that none of the indications favorably biased the results.

### Example 2: Exemplary IHC Protocols for Detecting MAGE-A4 Protein

This example describes exemplary IHC methods as provided herein, and demonstrates the efficacy of MAGE-A4-detecting IHC protocols as provided herein.

This study was conducted on FFPE myxoid/round cell liposarcoma (MRCLS), synovial sarcoma (SS), and non-small cell lung cancer (sqNSCLC) squamous cell carcinoma specimens stained with MAGE-A4 IHC using the EnVision FLEX visualization system on ALUTOSTAINER LINK 48™. WSI scoring was performed by qualified observers using the Aperio and ImageScope software.

### Reagents

SK032 MAGE-A4 IHC contains optimized reagents and protocol required to complete an IHC staining procedure using AUTOSTAINER LINK 48™ and PT Link Pre-treatment Module™ (https://www.agilent.com/en/product/pt-link-for-pre-treatment/pt-link-accessories/pt-link-pre-treatment-module-for-tissue-specimens-76929). Following incubation with the primary monoclonal antibody to MAGE-A4 or the Negative Control Reagent (NCR), specimens were incubated with a ready-to-use visualization reagent consisting of secondary antibody molecules and horseradish peroxidase (HRP) molecules coupled to a dextran polymer backbone. The enzymatic conversion of the subsequently added chromogen results in precipitation of a visible reaction product at the site of the antigen. The specimen was then counterstained and coverslipped. Results were interpreted using a light microscope.

TABLE 4
Assay components for exemplary
MAGE-A4-detecting IHC protocol

| Component | Component Description |
|---|---|
| Peroxidase-Blocking | Buffered solution containing hydrogen |
| Reagent | peroxide, detergent, and 0.015 mol/L |
| | sodium azide. |
| Mouse Monoclonal anti- | Monoclonal Mouse anti-MAGE-A4 in a |
| MAGE-A4 antibody, | buffered solution, containing |
| Clone OTI1F9 | stabilizing protein, and 0.015 mol/L |
| | sodium azide. |
| Negative Control | Monoclonal Mouse control IgG2a |
| Reagent (NCR) | antibody in a buffered solution, |
| | containing stabilizing protein, |
| | and 0.015 mol/L sodium azide. |
| Visualization | Dextran coupled with peroxidase |
| Reagent-HRP | molecules and goat secondary |
| | antibody molecules against rabbit |
| | and mouse immunoglobulins in a |
| | buffered solution containing stabilizing |
| | protein and an antimicrobial agent. |
| DAB + Substrate | Buffered solution, containing hydrogen |
| Buffer | peroxide and an antimicrobial agent. |
| DAB + Chromogen | 3,3′-diaminobenzidine |
| | tetrahydrochloride in organic solvent. |
| Target Retrieval | Buffered solution, containing detergent |
| Solution, High pH, 50X | and an antimicrobial agent. |

EnVision FLEX Hematoxylin (Dako K8008)
Automated coverslipping instrument or ClearMount (American MasterTech Scientific, MMCLE126), or equivalent mounting medium
Alcohol Reagent, Ethanol: absolute (Dako 100188) and 95%
Xylene (Dako 100941) or xylene substitute, i.e. Histoclear (Dako 100791)
EnVision FLEX Wash Buffer 20× (Dako K8007)
Distilled or deionized (reagent-grade water), or Dako reagent water (Dako PD00004)

### Reagent Preparation

Equilibrate kit reagents, buffers, and unstained slides to room temperature, 20-25° C.
Prepare 1× Wash Buffer by diluting 20× Wash Buffer in distilled or deionized (reagent-grade) water and mixing.
Discard Wash Buffer if it is cloudy in appearance.
Prepare 1× High pH Target Retrieval Solution (TRS) by diluting 50× High pH Target Retrieval Solution in reagent-quality water and mixing.
   Prepare a sufficient quantity of High pH 1× Target Retrieval Solution by diluting Target Retrieval Solution, High pH (50×) 1:50 using distilled or deionized (reagent-grade) water. One 30 mL bottle of Target Retrieval Solution, High pH (50×), diluted 1:50 provided 1.5 L of 1× reagent, sufficient to fill one PT Link tank which treated up to 24 slides per use. TRS pH test should be performed to ensure 1×TRS is within reagent specifications (pH 9.0±0.2) prior to use.
Prepare DAB+ chromogen shortly before initiating AUTOSTAINER LINK 48™ run. To each 1 mL of Substrate Buffer add 1 drop of Liquid DAB+ chromogen.
   If using a bottle of 7.2 mL DAB+ Substrate Buffer, add 9 drops of DAB+ chromogen. Although the label states 7.2 mL, this is the useable volume and does not account for the “dead volume” (1.8 mL) in the bottle.
   The color of the Liquid DAB+ Chromogen in the bottle may vary from clear to lavender-brown. This will not affect the performance of this product. Dilute per the guidelines above. Addition of excess Liquid DAB+ Chromogen to the DAB+ Substrate Buffer will result in deterioration of the positive signal.

### Specimen Preparation

All specimens were fixed in neutral buffered formalin and embedded in paraffin blocks. FFPE blocks were cut at 4 μm, mounted onto charged microscope slides, and placed at 58 (±2) ° C. for approximately 1 hour within 18 hours of sectioning, then cooled to room temperature (RT). Cut sections were kept at 2-8° C. until use.

### Staining Procedure

### Deparaffinization/Rehydration/Target Retrieval Procedure: PT Link

All specimens were processed using the PT Link (Code PT100/PT101 and/or PT200) with the Preheat and Cool temperature set to 65° C. All specimens were then processed using a PT Link set Heat temperature to 97° C. for 20 minutes.
Filled PT Link tanks with 1.5 L per tank of Target Retrieval Solution, High pH, 1× working solution to cover the tissue sections.
Preheated the Target Retrieval Solution to 65° C.
Immersed Autostainer racks containing mounted, FFPE tissue sections into the pre-heated Target Retrieval Solution, High pH, (1× working solution) in PT Link tank. Incubate for 20 minutes at 97° C.
As soon as target retrieval incubation time had been completed and the temperature had cooled to 65° C., remove each Autostainer slide rack with the slides from the PT Link tank and immediately placed the Autostainer rack with slides into a tank (e.g., PT Link Rinse Station, Code PT109) containing 1×, room temperature EnVision FLEX Wash Buffer (Code K8007).
Immersed slides in diluted, room temperature Wash Buffer for 5 minutes.

After deparaffinization, rehydration and target retrieval (3-in-1) procedure (specimen pre-treatment), the Autostainer racks with slides were placed on AUTOSTAINER LINK 48™. The AUTOSTAINER LINK 48™ instrument performed the staining process automatically using pre-programmed protocol by applying the appropriate reagent, monitoring the incubation time and rinsing slides between reagents. Stained slides for all indications were scored by qualified observers.

### Data Interpretation

The following information was captured during observer reads:
MAGE-A4 slide: Percent positive MAGE-A4 score at each intensity bin (0 to 3+)
Diagnostic outcome
   Positive: MAGE-A4 TIPS≥2+ staining intensity at ≥75% cutoff
   Negative: MAGE-A4 TIPS≥2+ staining intensity at ≤75% cutoff
Average IHC staining intesity
NCR slide: Pass/Fail
   The Negative Control Reagent (NCR) stained slide must not display specific staining. Non-specific staining at ≤1+ staining intensity is acceptable.
   Specific staining is defined as nuclear and/or cytoplasmic staining in tumor cells and non-specific staining is defined as staining of non-tumor tissue elements. If the NCR specimen displays specific staining at any intensity, or non-specific staining at >1+ intensity, DO NOT proceed with evaluation of the MAGE-A4 stained slide
Comments, if applicable

### Specimen

Eighty specimens (MRCLS and/or SS, sqNSCLC) demonstrating a range of MAGE-A4 expression were selected in the study. Efforts were made to ensure a balanced positive/negative specimen set around the MAGE-A4 TIPS≥2+ staining intensity at ≥75% cutoff with approximately 20-25% of samples falling within the near cutoff range (65-85%) at 2+ or greater staining intensity. Stained slides were selected from previously performed screening runs and the methods described in the protocol were followed.

### Equipment for Digital Scoring

Computer System with Windows 7 or higher operating system. The minimum computer monitor requirements must support 24-bit color depth and display resolution 1680(h)×1050(v) with a screen size of 24-inches according to the Aperio System Requirements. Computer monitor contrast and color control settings must be set to default factory settings.

### Procedure

### Test Objective

The objective of this test was to compare glass and WSI diagnostic status by observers scoring the same set of slides on two platforms a bright-field light microscope and a high-resolution computer monitor. Scanning of glass slides was performed using the APERIO AT2 SCANNER™.

### Initial WSI Requirements

Operator(s) of the APERIO AT2™ were trained prior to scanning glass slides.

Observer(s) viewing slides in ESLIDE MANAGER™ and IMAGESCOPE™ software were trained to navigate WSI using the viewing software.

### Scoring Guidelines:

Stained slides for all indications were scored by qualified observers

### WSI Scanning Procedure

Before scanning slides, slides were inspected for dust and/or residues. If any residues were present, operator cleaned the slide with a Kimwipe.

Scanning of slides using the APERIO AT2 SCANNER™. The operator reviewed WSIs for complete tissue area coverage and image clarity. If a WSI did not appear satisfactory to the operator, the operator re-scanned the associated glass slide prior to any scoring evaluations by observers.

### Blinding and Randomizing

Per CAP pathology guidelines (Pantanowitz et al., Arch. Pathol. Lab. Med. 137(12): 1710-1722), blinding and randomization of stained sections were performed to avoid bias during scoring of stained sections. Slides were evaluated by each observer in a random order according to a randomization key. Score sheets based on randomized slide order were prepared for recording results and certified observers were instructed to read slides only in the order they are presented. WSI reads were performed using the function in the APERIO IMAGESCOPE™ viewing software that blinds the observer to the slide label.

### Scoring of Glass Slides and WSI

Scoring of MAGE-A4 slides were performed by three qualified observers. Scoring was performed manually using a light microscope and digitally by viewing whole slide images (WSIs) in the APERIO IMAGESCOPE™ viewing software.

### Concordance Test Procedure

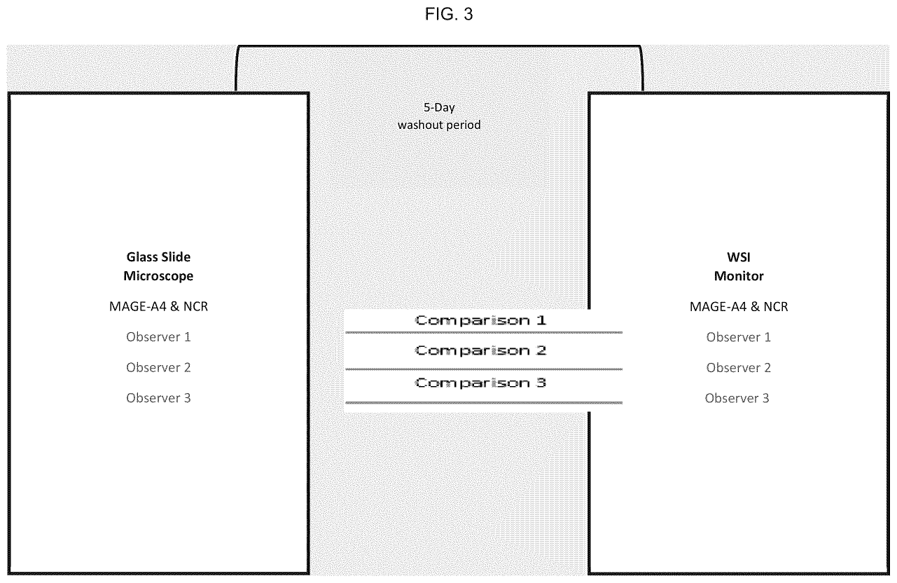

Three observers scored 80 blinded and randomized specimens manually on glass slides using a bright-field light microscope for read 1. After a minimum 5-day washout, the same observers scored WSIs of the same set of specimens using APERIO IMAGESCOPE™ viewing software for read 2, as shown in FIG. 3. Glass and WSI reads may have occurred in the reverse order. Reads were split into multiple scoring sessions as long as the minimum washout period was met between the glass and whole slide image (WSI) reads for a given specimen.

### Concordance Analysis

Since this study has a reference (result from glass slide scoring), negative, positive and overall percent agreements (NPA, PPA, OA) were calculated by comparing the WSI score to the glass score matched by observer and specimen, and the two-sided 95% confidence interval was calculated using the bootstrap method. MAGE-A4 TIPS at ≥2+ staining intensity ≥75% at cut-off applied to the glass scores was used to define the reference for agreement analyses. In the event that NPA, PPA, or OA exactly equal 100%, bootstrap is known to fail, so Wilson score confidence intervals were computed.

### Acceptance Criteria

The lower bound of the two-sided 95% confidence intervals of NPA, PPA and OA must each be at least 85% when all data from the three observers and indications are pooled. Acceptance criteria applies to the MAGE-A4 TIPS at ≥2+ staining intensity ≥75% cut-off.

Additional analysis, including per-observer or per-indication analysis, were not subject to acceptance criteria

### Results

Data from all indications were combined for analysis. Analysis was performed per indication for exploratory purposes and shown in Table 6.

TABLE 6
Summary of MRCLS, NSCLC, SS Glass vs WSI
SK032 Glass vs WSI, MAGE-A4 TIPS at ≥2 + staining
intensity ≥ 75% Cut-off
Agreement Summary

| | | | 95% Confidence Interval | |
|---|---|---|---|---|
| | Performance | Point | Lower-bound: | Upper-bound: |
| Indication | Criteria | Estimate | 2.5% | 97.5% |
| MRCLS, | NPA | 94.9% | 90.4% | 99.0% |
| NSCLC, | PPA | 97.2% | 94.3% | 99.3% |
| SS combined | OA | 96.2% | 93.8% | 98.3% |
| MRCLS | NPA | 100.0%* | 75.8% | 100.0% |
| | PPA | N/A | N/A | N/A |
| | OA | 100.0%* | 75.8% | 100.0% |
| NSCLC | NPA | 94.3% | 88.2% | 100.0% |
| | PPA | 97.0% | 92.9% | 100.0% |
| | OA | 95.8% | 92.5% | 98.3% |
| SS | NPA | 94.1% | 84.4% | 100.0% |
| | PPA | 97.3% | 93.2% | 100.0% |
| | OA | 96.3% | 92.6% | 99.1% |

*The bootstrap method cannot compute confidence bounds if 100% agreement is observed. The Wilson Score limits are used to calculate confidence intervals for agreement parameters with point estimates equal to 100%.

Continuous score plots were generated to compare the MAGE-A4 TIPS at ≥2+ staining intensity ≥75% scores between the conditions for the paired Glass and WSI scores for each observer and indication.

### Conclusions

The lower bound of the two-sided 95% confidence intervals of NPA, PPA and OA was at least 85% when glass and WSI comparisons from all three observers and indications were pooled. Equivalence has been established between scoring of glass slides using light microscopy and digital WSI when using the WSI system and scoring system specified in this report for SS, MRCLS, and squamous NSCLC slides stained with the SK032 MAGE-A4 IHC assay and interpreted at the MAGE-A4 TIPS at ≥2+ staining intensity ≥75% cut-off.

In addition to running the analysis on all indications pooled, the per-indication, per-observer, and per-indication-observer level were analyzed for informational purposes only. Based on the per-indication analysis, all indications showed high agreement in both negative and positive sample pools, so none seem to be heavily biasing the pooled indication results.

A number of embodiments of the invention have been described. Nevertheless, it can be understood that various modifications may be made without departing from the spirit and scope of the invention. Accordingly, other embodiments are within the scope of the following claims.

What is claimed is:

1. An immunohistochemistry (IHC) method for determining and scoring the extent of cellular expression of Melanoma Associated Antigen Gene-A4 (MAGE-A4) in a tissue sample, comprising:
(a) staining a tissue sample with an antibody which specifically binds to MAGE-A4;
(b) determining a total number of viable tumor or cancer cells having MAGE-A4 staining, and determining a total number of staining and non-staining viable tumor or cancer cells in at least a portion of the tissue sample,
wherein a tumor or cancer cell is counted as positively stained with
anti-MAGE-A4 antibody if there is cytoplasmic and/or nuclear MAGE-A4 staining at any intensity above a defined threshold; and
(c) determining a MAGE-A4 tumor intensity proportion score (MAGE-A4 TIPS),
wherein the MAGE-A4 TIPS is the number of MAGE-A4 staining viable tumor or cancer cells found in the tissue sample divided by the total number of staining and non-staining viable tumor or cancer cells, multiplied by 100.

2. The method of claim 1, wherein the defined threshold comprises:
a 1+ positive staining intensity evaluated at a high magnification;
a 2+ positive staining intensity evaluated at a medium magnification; or
a 3+ positive staining intensity evaluated at a low magnification.

3. The method of claim 2, wherein the low magnification is at least about 4× magnification; the medium magnification is at least about 10× magnification; and the high magnification is at least about 20× magnification or at least about 40× magnification.

4. The method of claim 2, wherein the MAGE-A4 TIPS comprises the number of MAGE-A4 viable tumor or cancer cells staining at a 2+ or greater positive staining intensity divided by the total number of staining and non-staining viable tumor or cancer cells, multiplied by 100.

5. The method of claim 2, wherein the MAGE-A4 TIPS comprises the number of MAGE-A4 viable tumor or cancer cells staining at a 1+ or greater positive staining intensity divided by the total number of staining and non-staining viable tumor or cancer cells, multiplied by 100.

6. The method of claim 1, wherein a MAGE-A4 TIPS of about 70% or greater indicates a positive diagnostic status of the tissue sample.

7. The method of claim 6, wherein the MAGE-A4 TIPS is about 75% or greater, about 80% or greater, or about 90% or greater.

8. The method of claim 1, wherein a MAGE-A4 TIPS of about 5% or greater indicates a positive diagnostic status of the tissue sample.

9. The method of claim 8, wherein a MAGE-A4 TIPS of about 5% or greater comprises the number of MAGE-A4 viable tumor or cancer cells staining at a 1+ or greater positive staining intensity; or a MAGE-A4 TIPS of about 10% or greater comprises the number of MAGE-A4 viable tumor or cancer cells staining at a 2+ or greater positive staining intensity.

10. The method of claim 1, wherein a section or portion of the tissue sample is prepared on a slide, a microscope slide, or equivalent, and the section or portion of the tissue sample is stained on the slide.

11. The method of claim 1, wherein the antibody comprises a monoclonal mouse anti-MAGE-A4 antibody.

12. The method of claim 11, wherein the monoclonal mouse anti-MAGE-A4 antibody comprises monoclonal mouse anti-MAGE-A4 clone OTI1F9, an antibody having a substantially similar affinity for MAGE-A4 as a clone OTI1F9 antibody, monoclonal mouse anti-MAGE-A4 clone 57B, monoclonal mouse anti-MAGE-A4 clone 6C1, monoclonal mouse anti-MAGE-A4 clone CPTC-MAGEA4-1, monoclonal mouse anti-MAGE-A4 clone CPTC-MAGEA4-2, monoclonal mouse anti-MAGE-A4 clone 8BA20, monoclonal mouse anti-MAGE-A4 clone 8BA21, monoclonal mouse anti-MAGE-A4 clone CBFYM-1312, or monoclonal mouse anti-MAGE-A4 clone 9A7.

13. The method of claim 1, wherein:
(a) the tissue sample comprises a formalin-fixed, paraffin-embedded (FFPE) specimen;
(b) the FFPE specimen comprises a cancer specimen stained on an automated IHC platform;
(c) the section of the tissue sample is prepared by a protocol comprising fixation in about 10% neutral buffered formalin for a time period of about 6 hours to about 72 hours;
(d) the tumor or cancer is a synovial sarcoma, a myxoid/round cell liposarcoma, a high grade myxoid liposarcoma, a low grade myxoid liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer;
(e) the tissue sample is derived from a needle biopsy sample, a fine-needle aspirate, a cytology specimen, or a bone decalcification;
(f) positive staining is determined using a bright-field light microscope, a microscope objective, a computer monitor and imaging software, or a combination thereof; and/or
(g) the imaging software comprises whole slide imaging software.

14. A method for diagnosing a tumor or a cancer by determining if a tissue sample is positive for expression of Melanoma Associated Antigen Gene-A4 (MAGE-A4), comprising:
determining a MAGE-A4 diagnostic status in a tissue sample by a method of claim 1, wherein a MAGE-A4 tumor intensity proportion score (MAGE-A4 TIPS) of about 70% or greater of tumor cancer cells having MAGE-A4 nuclear and/or cytoplasmic staining at an intensity of 2+ or greater is diagnostically positive.

15. The method of claim 20, wherein the tumor or cancer is a synovial sarcoma, a myxoid/round cell liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

16. A method for treating or ameliorating a tumor or a cancer in a patient, comprising determining and scoring the amount of MAGE-A4 in a tissue sample from the patient using a method of claim 1, wherein if the tissue sample is determined or scored to have a high or a diagnostically positive MAGE-A4 score, the patient is treated with a cancer therapeutic to which the patient is likely to respond favorably.

17. The method of claim 16, wherein the tumor or cancer is a synovial sarcoma, a myxoid/round cell liposarcoma, a head and neck cancer, a melanoma an esophageal cancer, a gastric cancer, a colorectal cancer, a lung cancer, a non-small cell lung cancer, a colon cancer, a breast cancer, an ovarian cancer, an endometrial cancer, a cervical cancer, a salivary gland cancer, a prostate cancer, a liver cancer, or a urothelial cancer.

18. The method of claim 16, wherein the cancer therapeutic comprises administration to the patient an anti-cancer drug or an anti-cancer therapy, and optionally the anti-cancer therapy comprises an immunotherapy, a monoclonal antibody therapy, an adoptive cell therapy (ACT), a T-cell receptor (TCR) therapy, or a chimeric antigen receptor (CAR) T-cell therapy.

19. A kit comprising an antibody which specifically binds to MAGE-A4 and MAGE-A4 scoring guidelines as set forth in a method of claim 1.

20. A method for assessing the extent of MAGE-A4 expression comprising:
contacting a sample or a portion thereof comprising cancer or tumor cells from an individual with an antibody or a portion thereof which specifically binds to MAGE-A4; and
determining a MAGE-A4 tumor intensity proportion score (MAGE-A4 TIPS) by dividing the number of MAGE-A4 staining viable tumor or cancer cells in the sample or portion thereof specifically bound by the antibody with the total number of staining and non-staining viable cancer or tumor cells and multiplying the result by 100, thereby obtaining the MAGE-A4 TIPS.
